(19) United States
(12) Patent Application Publication
(10) Pub. No.: US 2010/0240749 A9
(43) Pub. Date: Sep. 23, 2010

(54) ANESTHETIC SPRAY COMPOSITION

(75) Inventors: Eric G. Spengler, Ridgefield, CT (US); Michael J. Borja, Keyport, NJ (US)

Correspondence Address: FITZPATRICK CELLA HARPER & SCINTO, 1290 Avenue of the Americas, NEW YORK, NY, 10104-3800, US

(73) Assignee: COMBE INCORPORATED, White Plains, NY (US)

(21) Appl. No.: 12/040,367

(22) Filed: Feb. 29, 2008

Related U.S. Application Data
(60) Provisional application No. 60/892,764, filed on Mar. 2, 2007.

(51) Int. Cl. A61K 31/24; A61P 29/00

(52) U.S. Cl. 514/536

(57) ABSTRACT

An anesthetic spray composition suitable for application to the back of the throat comprising a topical anesthetic and a mucoadhesive polymer.

This application claims the benefit of U.S. Provisional Application No. 60/892,764, filed Mar. 2, 2007, which is incorporated herein by reference.

### BACKGROUND OF THE INVENTION

1. Field of the Invention

The present invention relates generally to a non-prescription anesthetic spray composition, which can be used to temporarily relieve sore throats.

2. Description of Related Art

The over-the-counter (OTC) market has offered spray products for the treatment of sore throats for many years. The majority of these products utilize phenol as their active ingredient. Phenol is the simple alcohol derivative of benzene. Both of these chemicals are listed by the Environmental Protection Agency (EPA) as extremely hazardous.

Alternative sore throat sprays have been provided that contain Piper methysticum (Kava Kava) as its main active ingredient, in place of phenol, as disclosed in U.S. Pat. No. 6,159,473 to Watkins et al. Addition of Echinacea angustifolia, in combination with several other homeopathic ingredients, primarily plant extracts, is said to combine the analgesic effects of Kava Kava with the immune and antiseptic effects of Echinacea, and addresses the concomitant symptoms and acute pathology associated with sore throats, including hoarseness, sinus congestion, post-nasal drip, and the condition of the lining of the throat.

Additional active ingredients for sore throat sprays are listed in the OTC Oral Discomfort Monograph in 21 CFR 356. Of these ingredients, only Dyclonine HCl is widely used in commercial products. One such product is sold under the trademark Cepacol®. Other, non-OTC actives may contain Piper methysticum.

While conventional throat sprays provide temporary sore throat relief, there is a need for a spray composition, which has longer-lasting effect and which can be applied more precisely to the desired area in the back of the throat. In particular, current spray products are usually applied in a large dose (about 3 ml) and are intended to be swished around the mouth for 15 seconds or gargled. There is a need to eliminate that type of application by providing a spray composition, which can be directed to back of throat.

### SUMMARY OF THE INVENTION

To overcome the deficiencies of the prior art, the present invention provides an improved spray composition, which can be applied in a more controlled manner and remains effective for a longer period of time.

According to the present invention, the spray composition includes a topical anesthetic and a carrier, which includes a mucoadhesive polymer. The carrier may contain a solvent for the anesthetic, water and an emulsifier, particularly if the topical anesthetic is hydrophobic.

### BRIEF DESCRIPTION OF THE DRAWINGS

FIGS. 1-10 show particle sizes and distributions obtained as a result of the spray tests performed in accordance with Example 3.

FIGS. 11A-11C are photographs of spray patterns in accordance with Example 4.

### DESCRIPTION OF THE PREFERRED EMBODIMENTS

The anesthetic spray of the present invention contains a topical anesthetic as a main active ingredient. Preferably, this topical anesthetic is benzocaine. However, other topical anesthetics, such as lidocaine, lidocaine hydrochloride, tetracaine, hydrochloride, benzyl alcohol, dyclonine hydrochloride, hexylresorcinol, menthol, phenol preparations (phenol and phenolate sodium), salicyl alcohol, Kava Kava and the like, can be used. Any desired combination of active ingredients may also be used.

The composition also includes a mucoadhesive polymer. The mucoadhesive polymer increases the surface tension of the spray droplets that are sprayed into the oral cavity, which greatly changes the spray pattern, making it more regular and controlled. Specifically, the increase in the surface tension results in an increase in the size of the droplets (i.e., the droplets are less atomized). Thus, the spray pattern becomes narrower, and the sprayed material may be directed to the back of the throat as opposed to diffusing in the mouth.

In addition to changing the rheology of the droplets, the mucoadhesive polymer also improves the long-lasting effects of the spray. Specifically, the mucoadhesive is able to trap the anesthetic on the back of the throat for a longer period of time due to its mucoadhesive properties.

Examples of the mucoadhesive polymers that may be used in accordance with the present invention include, but are not limited to, Gantrez®, synthetic cellulose derivatives (e.g., sodium CMC, HPC, HPMC, HEPMC, etc.), natural gums (sodium alginate, karaya, carrageenan, xanthan, guar, chitosan, etc.), and synthetic polymers. Examples of synthetic polymers include polyvinyl pyrrolidone (PVP), polyvinyl pyrrolidone/vinyl acetate copolymer (PVP/VA), other copolymer, such BASF's KOLLIDON VA64, polyethylene oxide, PLURONIC® polymers, polyacrylic acid, polyacrylamides, polyvinyl alcohol and the like. Preferably, the anesthetic composition contains PVP.

Even small quantities of the mucoadhesive polymer can have a significant effect on the ability to apply the spray to the back of the throat. If the mucoadhesive polymer content is too large, the spray pattern becomes too concentrated, resulting in a dense spray stream, which may require an application of a large amount of the anesthetic to cover the desired area. The excess amount of the anesthetic composition may drip, which is likely to cause discomfort.

Preferably, the spray composition contains from about 0.01% to about 5% by weight of the mucoadhesive polymer. For these and other purposes, the PVP content is preferably from about 0.05% to about 0.5% by weight, more preferably from about 0.08% to about 0.3% by weight, yet more preferably from about 0.09% to about 0.2% or from about 0.09% to about 0.11% by weight. A sufficient amount of PVP is employed to provide satisfactory rheology-modifying and mucoadhesive characteristics to achieve the desired effect.

The carrier includes a solvent for the mucoadhesive polymer. One such solvent may be an alcohol. The alcohol may comprise from about 5 to about 45% by weight of the spray composition. Preferably, the alcohol content is from about 15% by weight to about 35% by weight. Ethanol or an approved specially denatured alcohol (e.g., SD-38F), which is commercially available from the Lyondell Chemical Company, may be used.

Another component of the carrier may be water. Water may comprise from about 15% to about 95% by weight of the spray composition. Preferably, the water content is from about 23% to about 70% by weight, more preferably from about 23% to about 29% by weight. Preferably, the water is deionized.

If the anesthetic agent is hydrophobic, such as benzocaine, and water is present in the composition, it is preferable to include another solubilizer and/or an emulsifier. The particular emulsifier used is selected on the basis of, for example, chemical compatibility, cost, as well as the shelf-life stability required.

Nonionic surfactants are preferred as the emulsifying/solubilizing agent in the spray composition. Anionic emulsifiers are less desirable since they generally provide an alkaline environment in which hydrophobic anesthetics, such as benzocaine, are less soluble. Cationic emulsifiers typically provide the desired acid environment, but are generally found to be irritants and are, therefore, also less desirable than the nonionic surfactants.

The number of suitable nonionic surfactants is legion; the most frequently used are esters of a polyethylene glycol having a molecular weight between about 200 and 600 particularly with fatty acids having 12 to 18 carbon atoms; esters of sorbitol with fatty acids having 12 to 18 carbon atoms (e.g., sorbitan stearate) and the polyethenoxy ethers of these esters, (e.g., Polysorbate-60); polyethenoxy ethers of alkanes and alkyl phosphates having 12 to 18 carbon atoms (Coceth-6 and PEG-75-Lanolin).

Polyoxyl 40 hydrogenated castor oil is a preferred emulsifier and solubilizing agent for use in the present invention. This agent is commercially available as Cremophor® RH 40 from BASF.

Other ingredients, which may be included in the spray composition include Eucalyptus globulus, Piper methysticum, Thymus vulgaris, Lycopodium clavatum, Phytolacca decandra, Capsicum annuum, Mentha piperita, and phosphorus, all in a base of purified water, sweeteners, and flavors.

Examples of natural sweeteners include, but are not limited to, fructose, sucrose, rice syrup, glucose, stevia, glycerin, honey, barley malt and the like. Other sweeteners include neotame, potassium acesulfame, aspartame, sodium saccharin, sucralose and the like. Examples of other types of flavors, both natural and artificial, include, but are not limited to, spearmint, cherry, wintergreen, thyme, fennel, anise and the like.

### Example 1

A throat spray composition as shown in Table 1 was prepared.

TABLE 1

| Ingredients | | |
|---|---|---|
| Trade Name | INCI Name | Weight (%) |
| Benzocaine, USP | Benzocaine | 5.00 |
| SDA-38B-72 | SD Alcohol 38B | 33.00 |
| Cremophor RH40 | PEG-40 hydrogenated | 4.00 |
| | castor oil | |
| Cherry flavor | Flavor | 0.2 |
| DI Water | Deionized Water | 24.55 |
| Glycerin, USP | Glycerin | 33.00 |
| Ace-K | Potassium Acesulfame | 0.10 |
| Neotame | Neotame | 0.05 |
| PVP K90 | PVP | 0.10 |
| | Total | 100.00 |

Benzocaine, alcohol, PEG-40 hydrogenated castor oil, the flavor and PVP are blended until a homogenous mixture (alcohol phase) is formed. Water, glycerin and the sweeteners are blended in a separate container until a homogenous mixture is formed (water phase). The aqueous phase is slowly added into alcohol phase. If needed, the resulting composition may be chilled and/or filtered.

Alternatively, benzocaine may be first dissolved in the alcohol, followed by PEG-40 hydrogenated castor oil and the flavor. The combination is mixed until clear. Sweeteners and PVP are slowly added and mixed until completely dissolved. Glycerin is then added and mixed until uniform. Water is added and mixed until the composition is clear and free of undissolved particles.

### Example 2

A throat spray composition as shown in Table 2 was prepared.

TABLE 2

| | Ingredients | |
|---|---|---|
| Trade Name | INCI Name | Weight (%) |
| Benzocaine, USP | Benzocaine | 5.00 |
| SDA-38F 8 | SD Alcohol 38F | 33.00 |
| Cremophor RH40 | PEG-40 hydrogenated | 3.00 |
| | castor oil | |
| Mint Flavors | Flavor | 0.65 |
| DI Water | Deionized Water | 25.17 |
| Glycerin, USP | Glycerin | 33.00 |
| Ace-K | Potassium Acesulfame | 0.05 |
| Neotame | Neotame | 0.03 |
| PVP K90 | PVP | 0.10 |
| | Total | 100.00 |

The method for preparing this composition is similar to that in Example 1.

### Example 3

Several anesthetic spray compositions were prepared and tested to determine the effects of the concentration of PVP on the spray pattern and droplet rheology. These compositions are shown in Table 3.

TABLE 3

| | | | C (% | D (% | E (% | F (% |
|---|---|---|---|---|---|---|
| Ingredients | A (% w/w) | B (% w/w) | w/w) | w/w) | w/w) | w/w) |
| Alcohol USP | 33.0 | 33.00 | 33.0 | 33.0 | 33.0 | 33.0 |
| 190 Proof | | | | | | |
| Cremophor | 3.0 | 3.00 | 3.0 | 3.0 | 3.0 | 3.0 |
| RH40 | | | | | | |
| Benzocaine, | 5.0 | 5.00 | 5.0 | 5.0 | 5.0 | 5.0 |
| USP | | | | | | |
| Glycerin, USP | 33.0 | 33.00 | 33.0 | 33.0 | 33.0 | 33.0 |
| PVP | 0.0 | 0.05 | 0.1 | 0.3 | 0.5 | 0.7 |
| Flavor | q.s. | q.s. | q.s. | q.s. | q.s. | q.s. |
| Sweetener | q.s. | q.s. | q.s. | q.s. | q.s. | q.s. |
| Water | 26.0 | 25.95 | 25.9 | 25.7 | 25.5 | 25.3 |
| Color | q.s. | q.s. | q.s. | q.s. | q.s. | q.s. |
| Total | 100.0 | 100.00 | 100.0 | 100.0 | 100.0 | 100.0 |

Compositions A-F were prepared by mixing the ingredients in the order listed in Table 3 and allowing each ingredient to dissolve or hydrate before adding the next component.

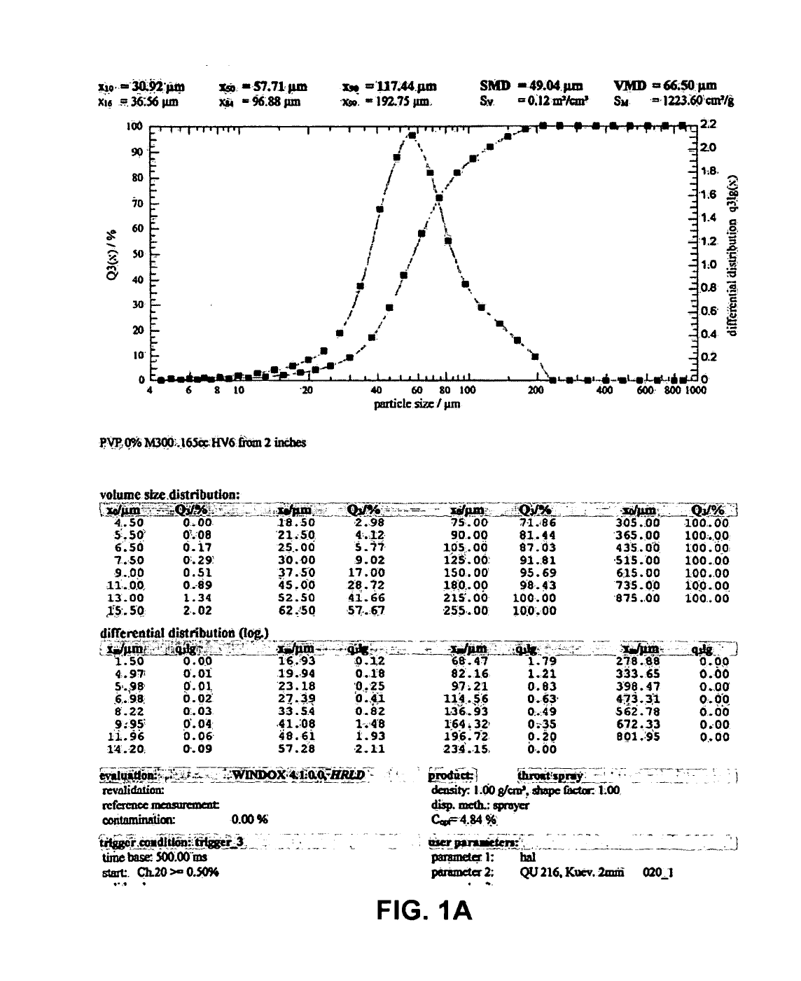

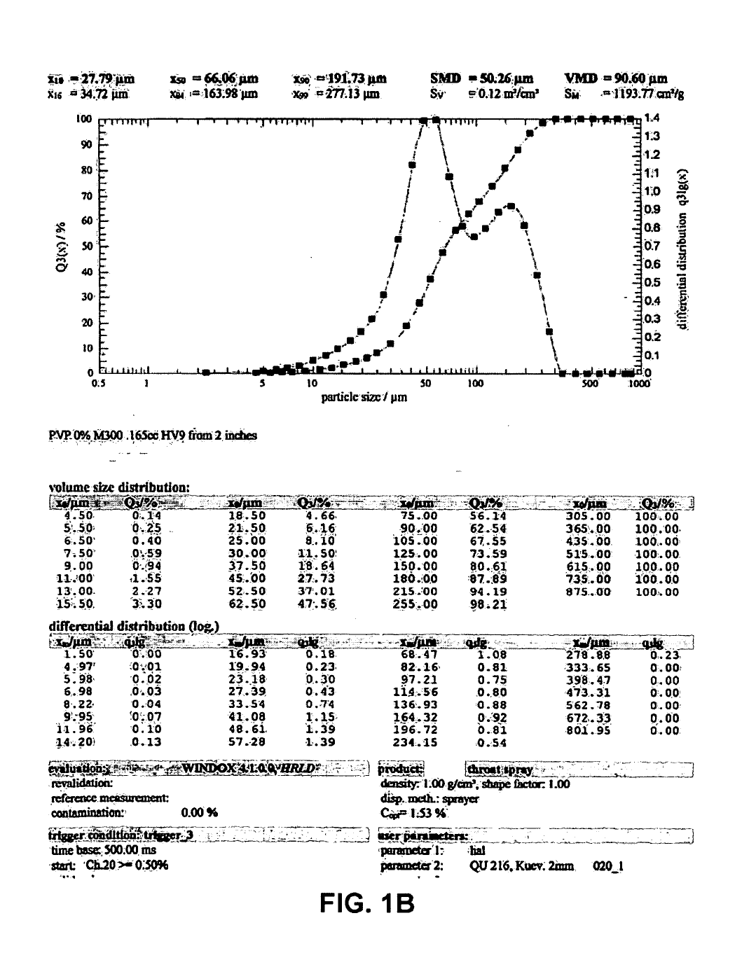

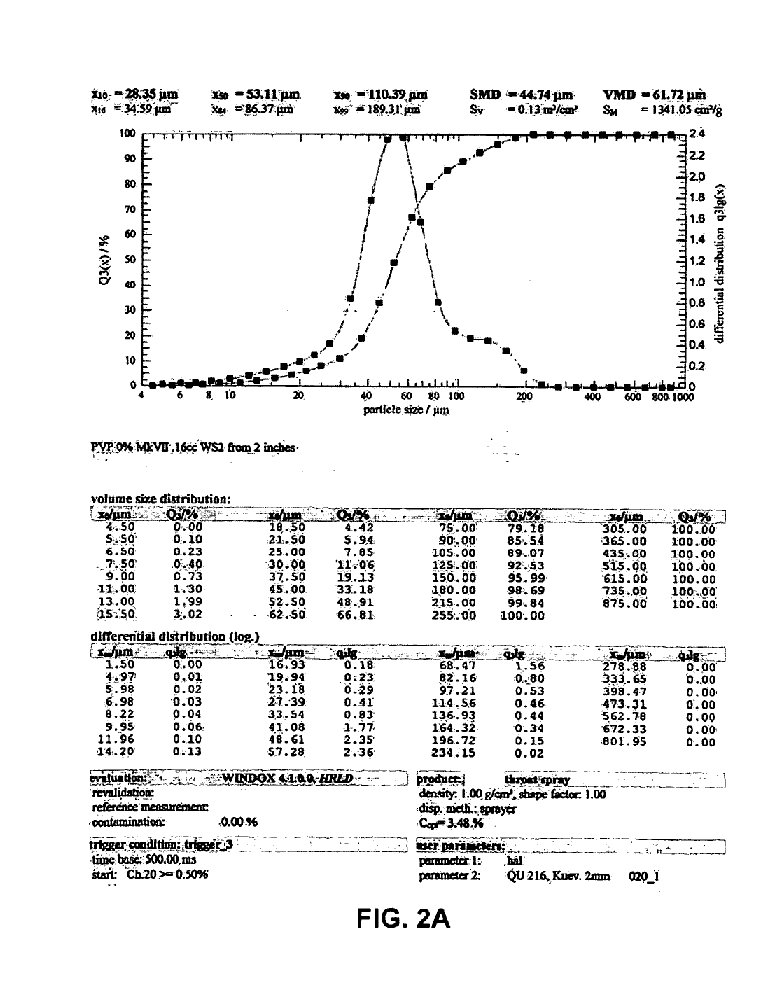

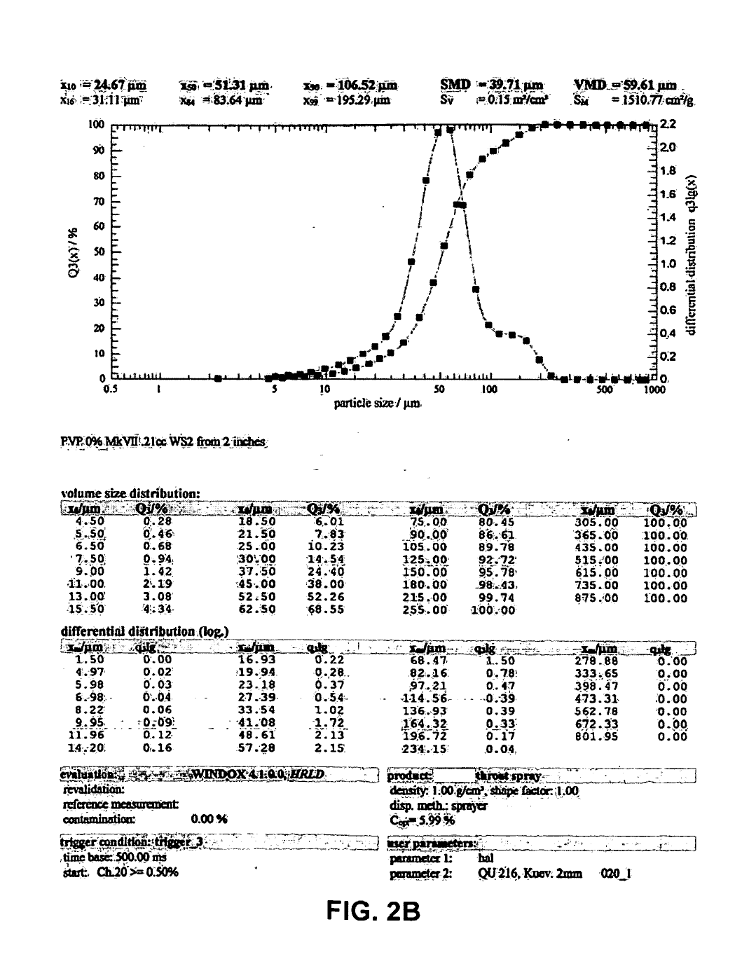

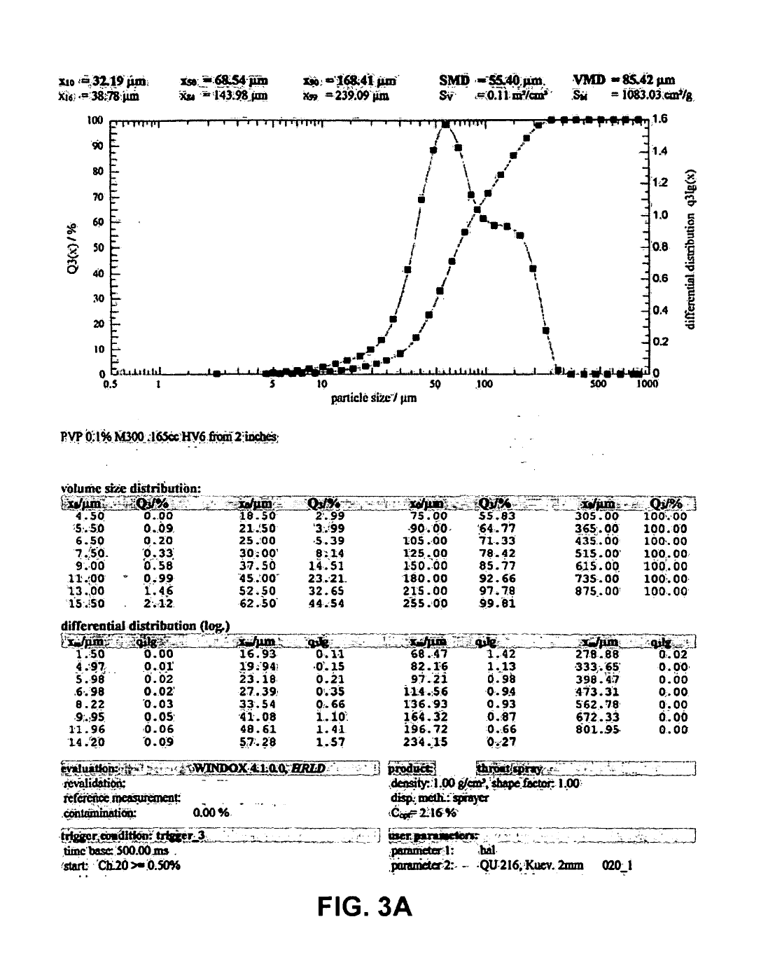

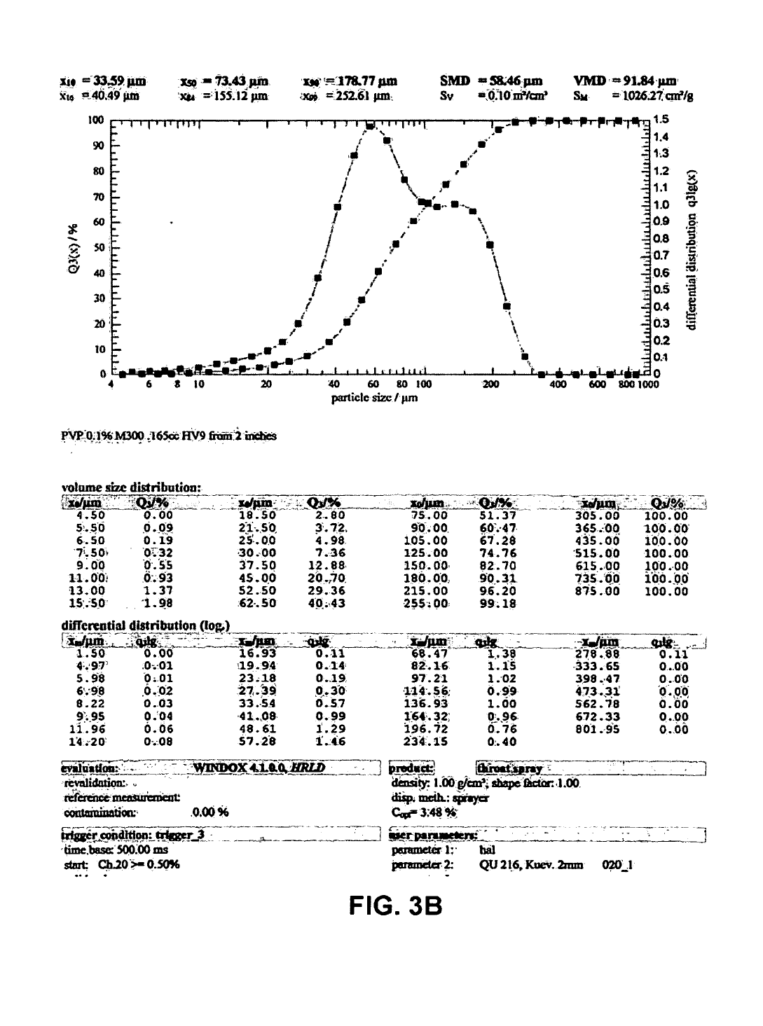

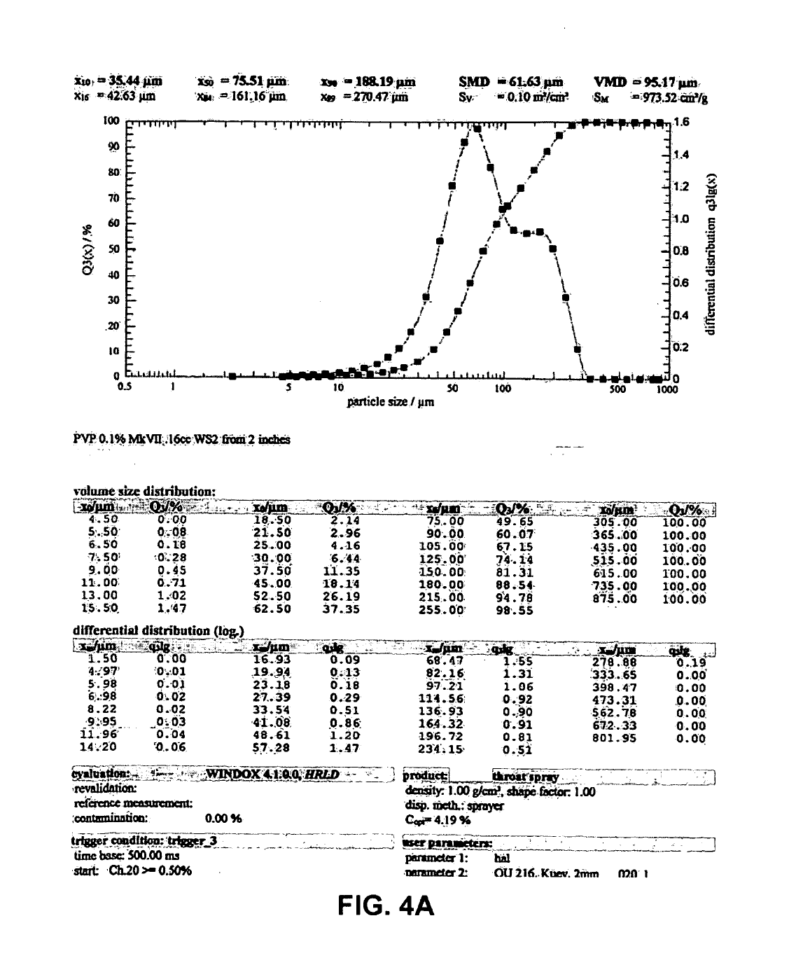

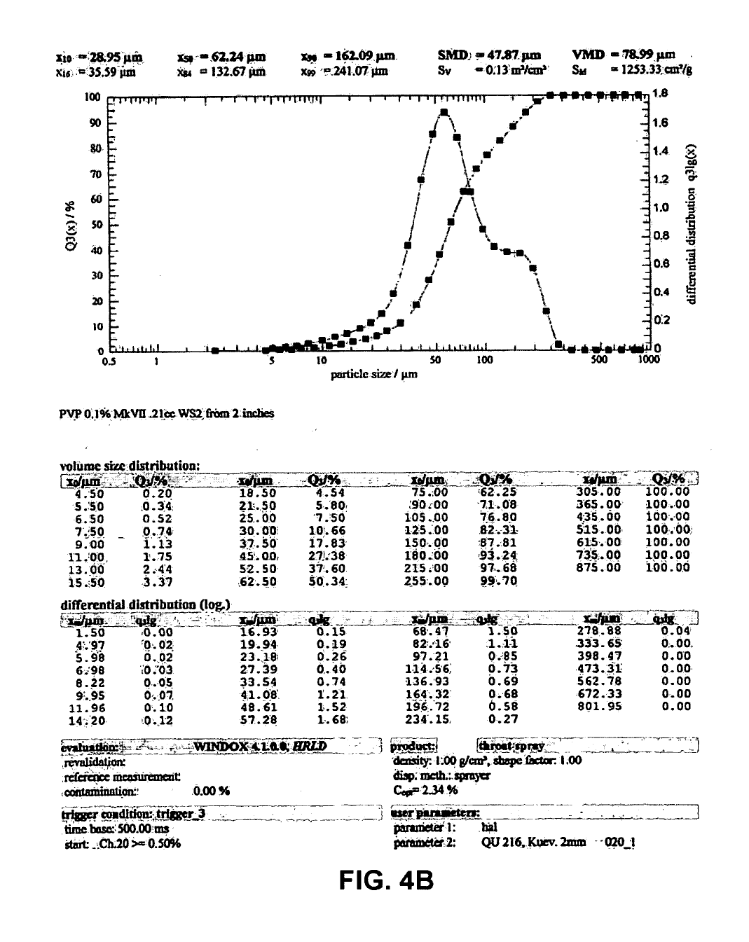

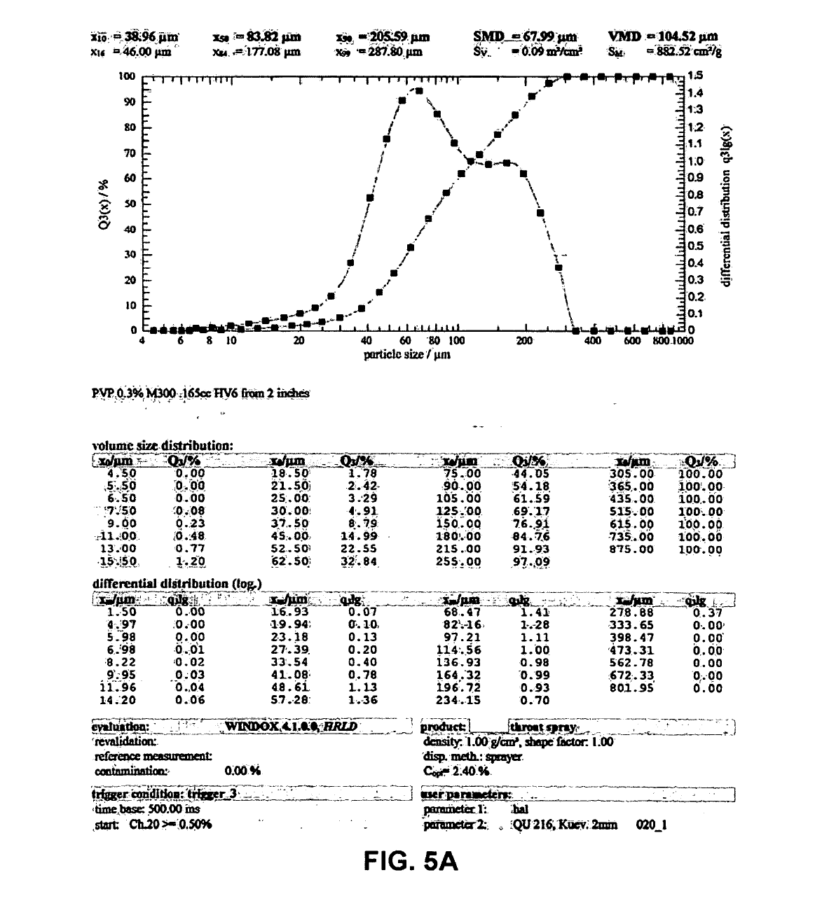

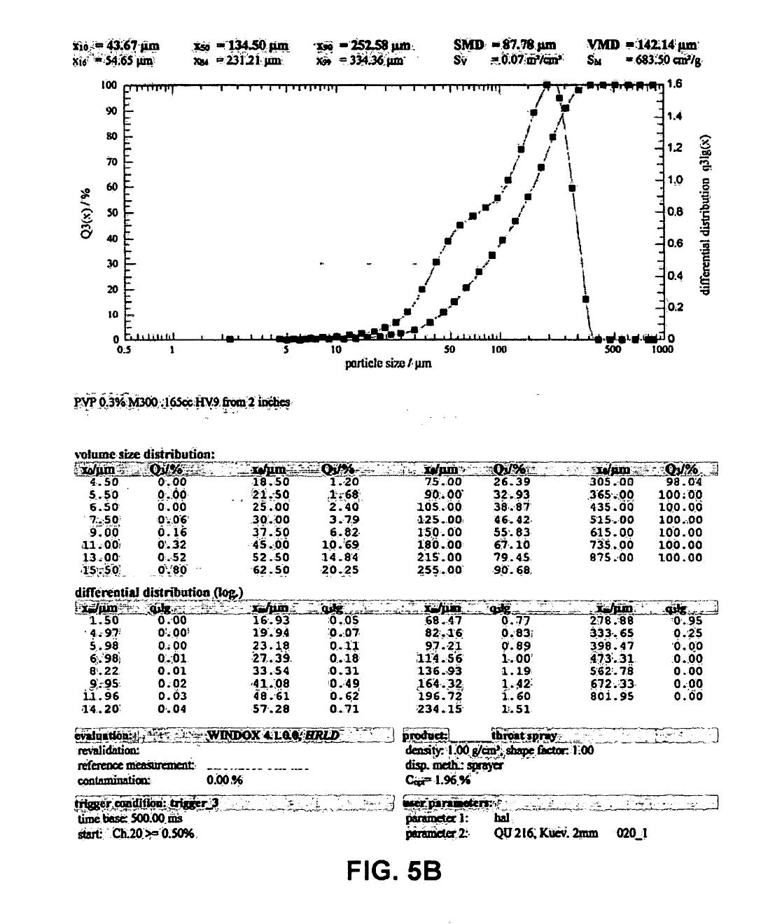

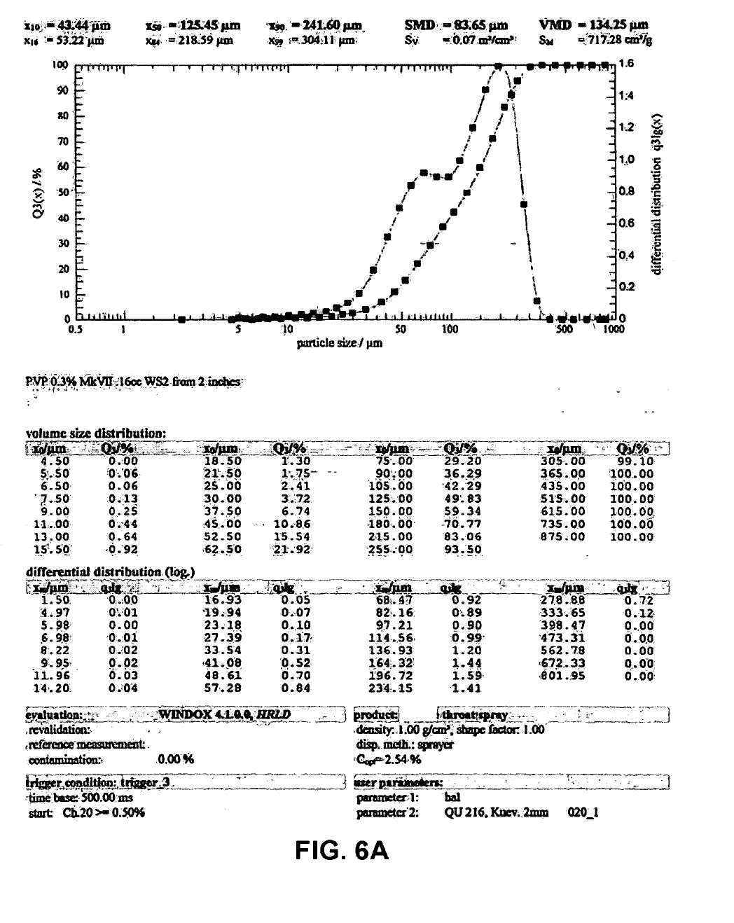

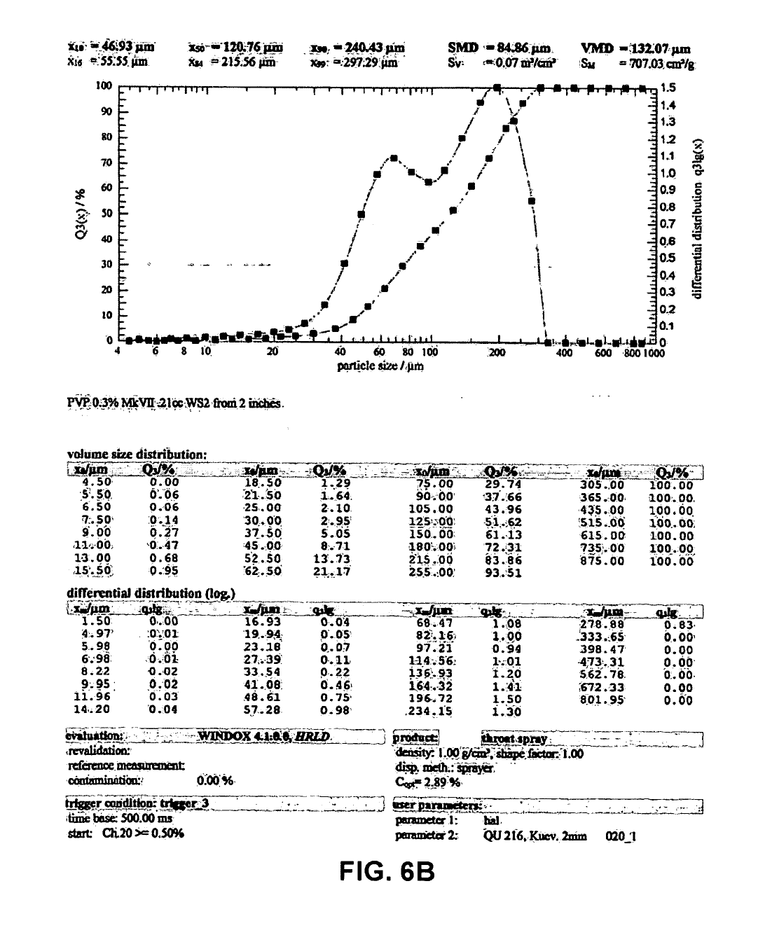

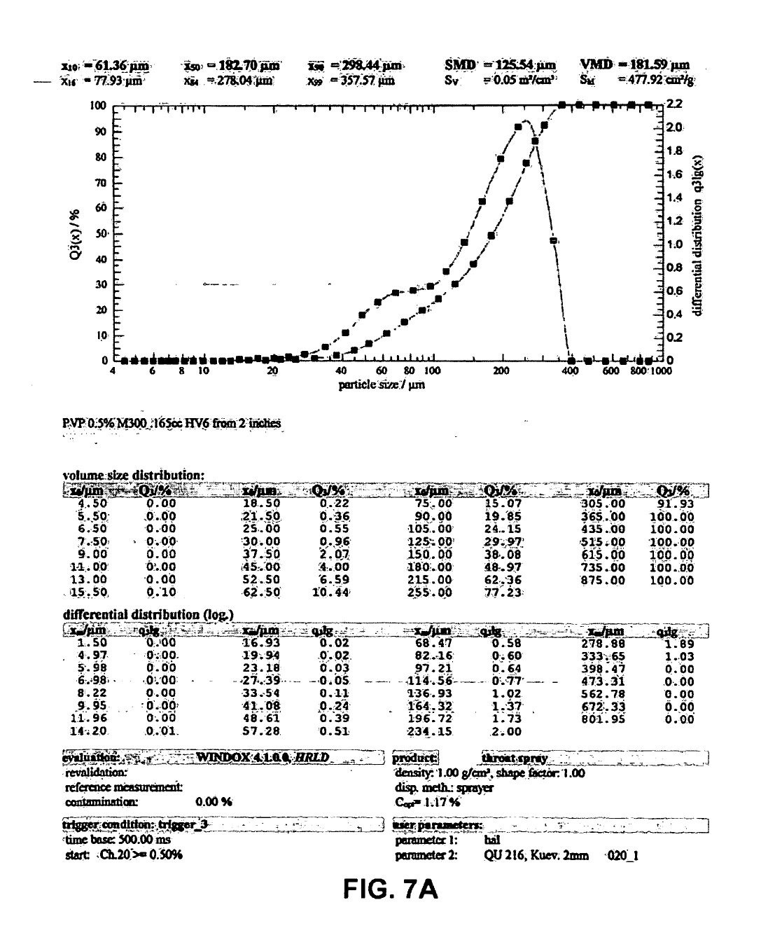

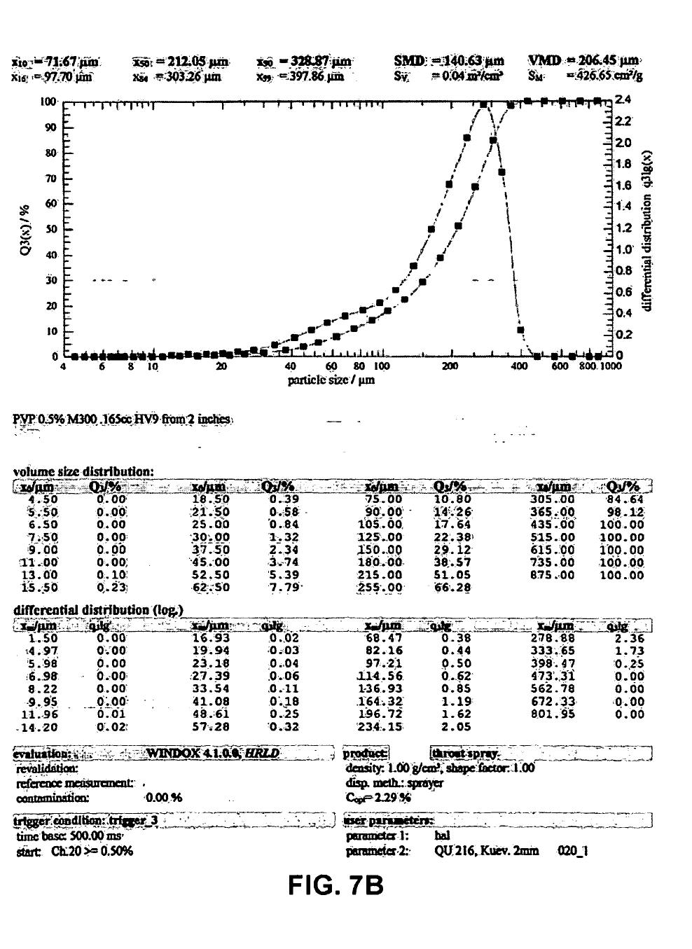

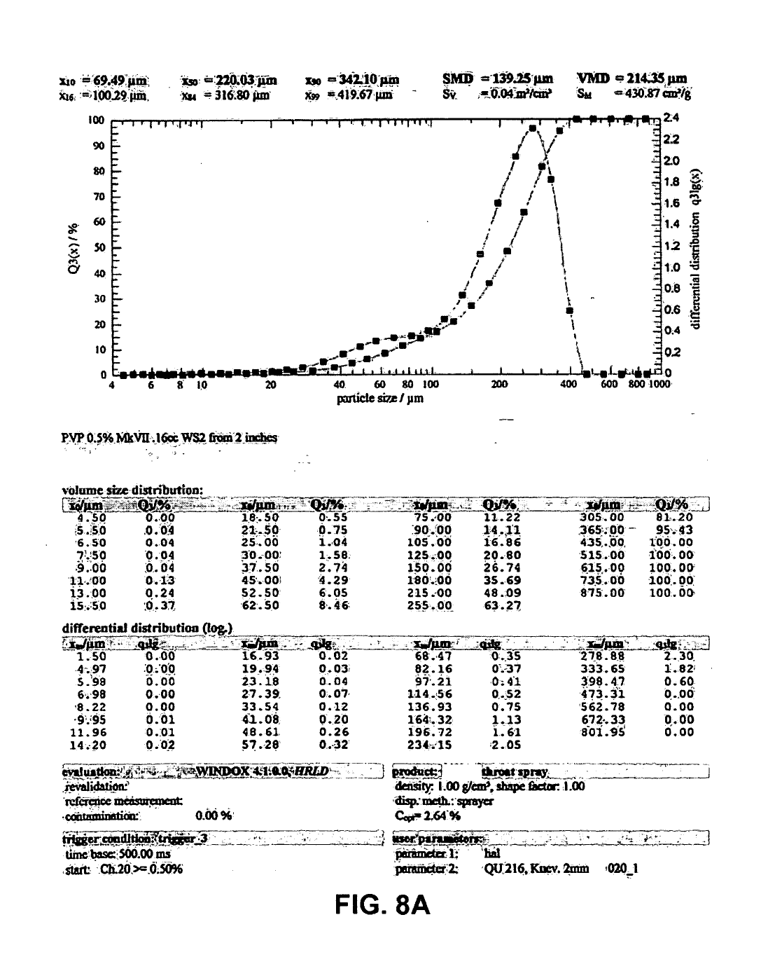

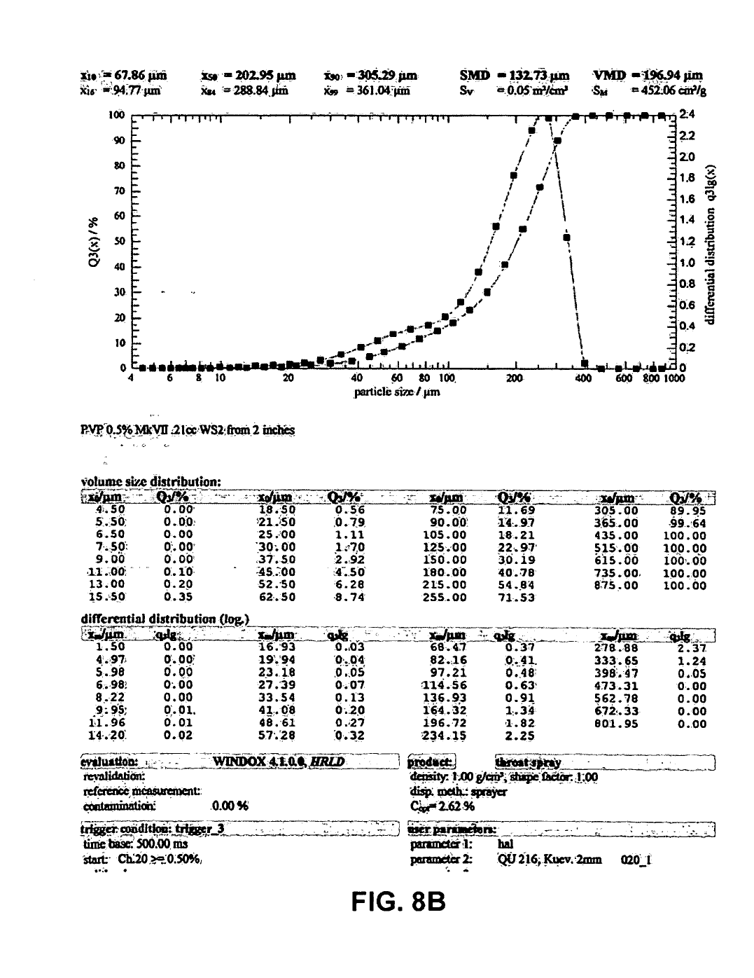

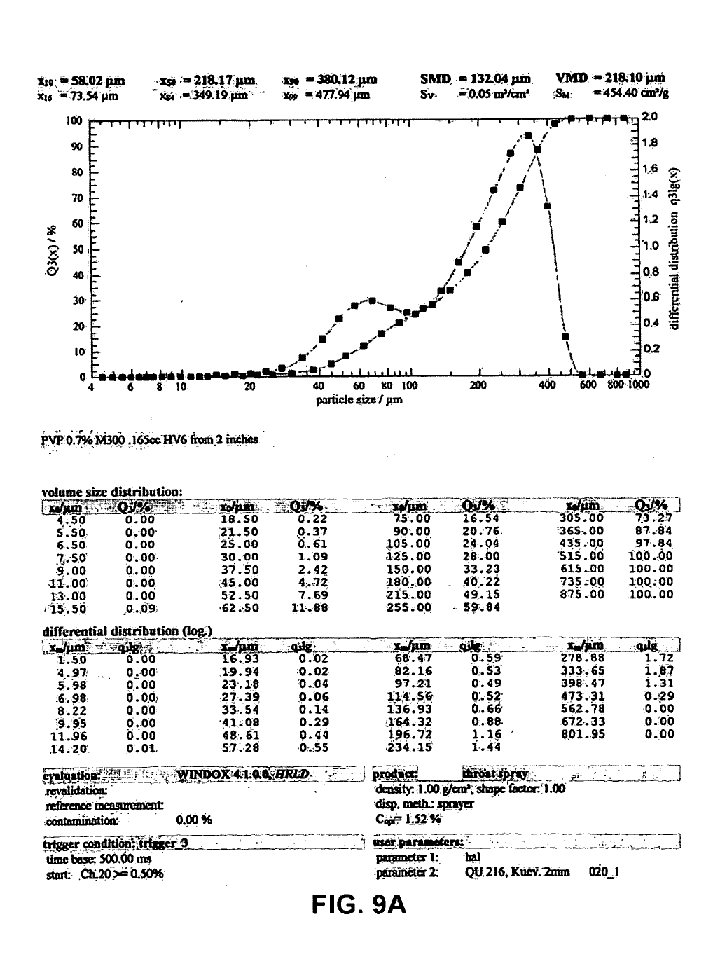

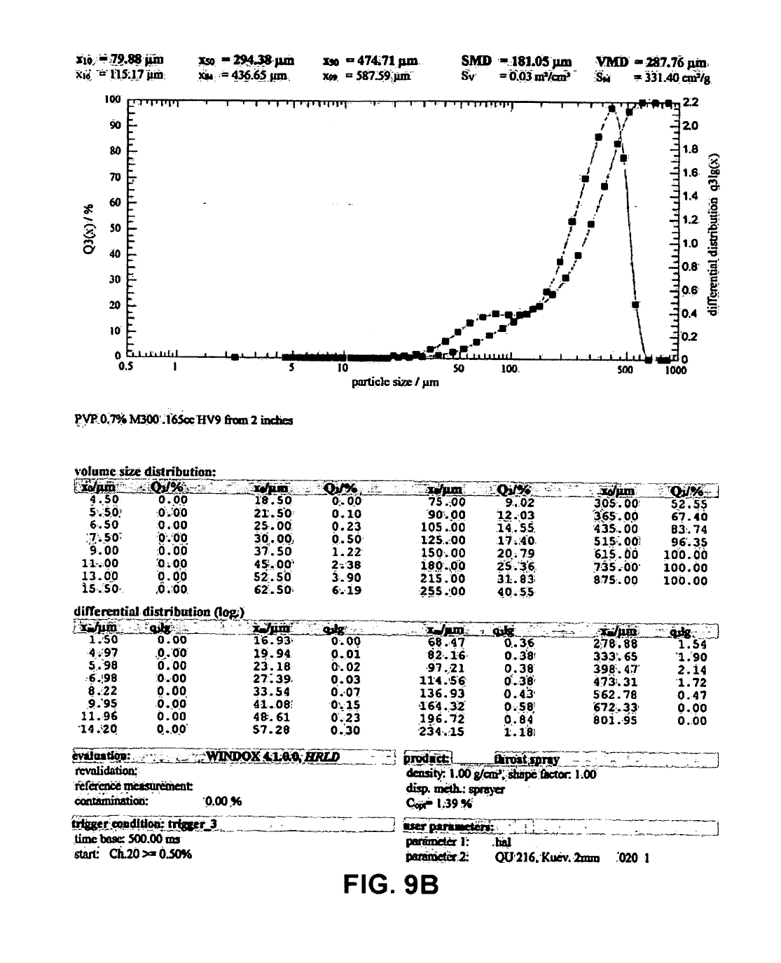

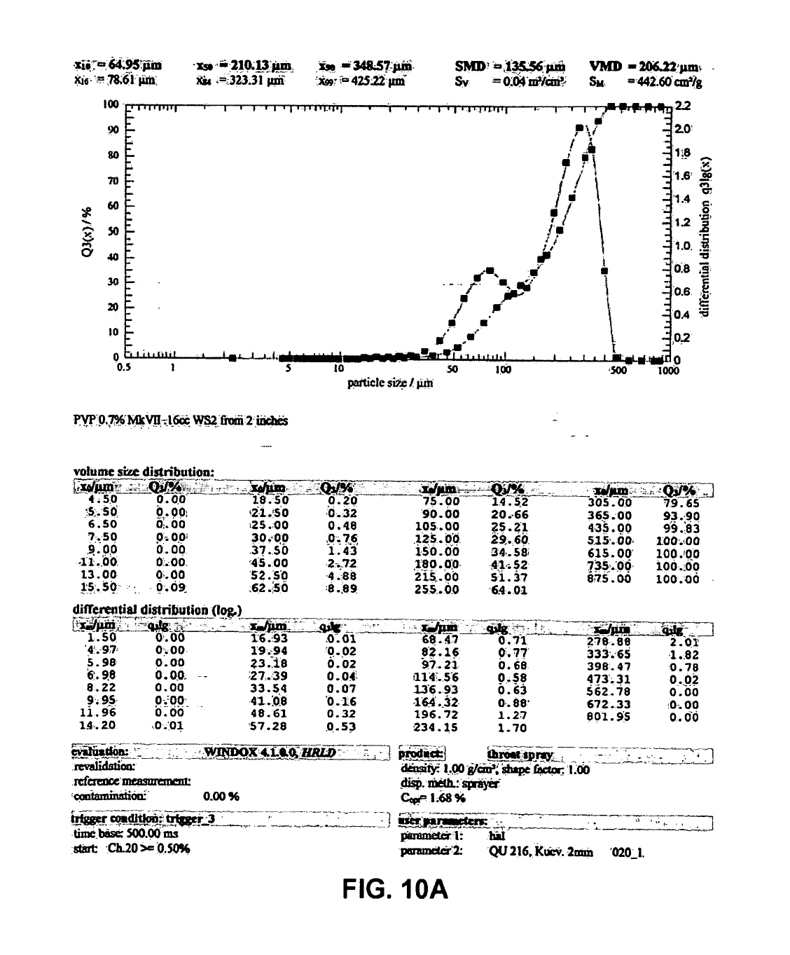

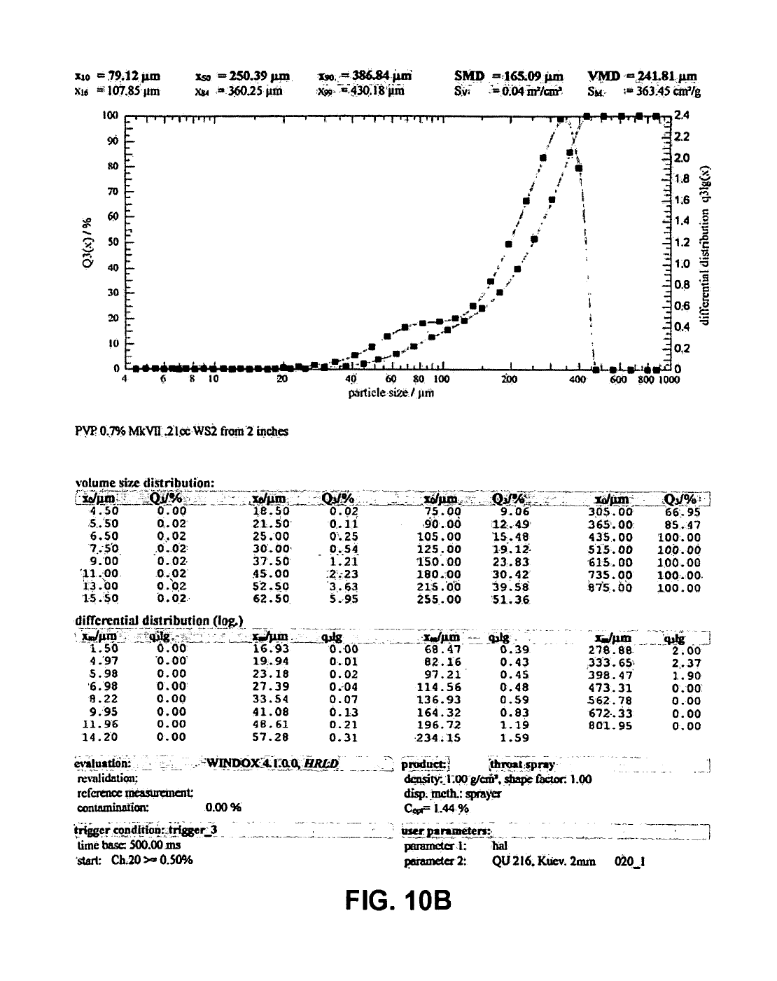

Compositions A and C-F were sprayed at a distance of two inches using two spray pump options available from MeadWestvaco Calmar. These were the M300 pump with two different inserts, HV6 and HV9, and the Mark VII pump with two different spray volumes, using the same insert, WS2. The results of the analysis of these spray tests are shown in Tables 4-8. The particle distribution for each of these tests is shown in FIGS. 1-10, as obtained using a HELOS Sympatech particle size analyzer.

TABLE 4
Composition A (0% w/w PVP)

| Spray | | Spray | | | | | | |
|---|---|---|---|---|---|---|---|---|
| Pump | Insert | (cc) | 10% | 50% | 90% | SMD | VMD | <15.5 μm |
| M300 | HV6 | 165 | 30.92 | 57.71 | 117.44 | 49.04 | 66.50 | 2.02% |
| M300 | HV9 | 165 | 27.79 | 66.06 | 191.73 | 50.26 | 90.60 | 3.30% |
| MkVII | WS2 | 16 | 28.35 | 53.11 | 110.39 | 44.74 | 61.72 | 3.02% |
| MkVII | WS2 | 21 | 24.67 | 51.31 | 106.52 | 39.71 | 59.61 | 4.34% |

TABLE 5
Composition C (0.1% w/w PVP)

| | | Spray | | | | | | |
|---|---|---|---|---|---|---|---|---|
| Spray | | Volume | | | | | | <15.5 |
| Pump | Insert | (cc) | 10% | 50% | 90% | SMD | VMD | μm |
| M300 | HV6 | 165 | 32.19 | 68.54 | 168.41 | 55.40 | 85.42 | 2.12% |
| M300 | HV9 | 165 | 33.59 | 73.43 | 178.77 | 58.46 | 91.84 | 1.98% |
| MkVII | WS2 | 16 | 35.44 | 75.51 | 188.19 | 61.63 | 95.17 | 1.47% |
| MkVII | WS2 | 21 | 28.95 | 62.24 | 162.09 | 47.87 | 78.99 | 3.37% |

TABLE 6
Composition D (0.3% w/w PVP)

| | | Spray | | | | | | |
|---|---|---|---|---|---|---|---|---|
| Spray Pump | Insert | Volume (cc) | 10% | 50% | 90% | SMD | VMD | <15.5 μm |
| M300 | HV6 | 165 | 38.96 | 83.82 | 205.59 | 67.99 | 104.52 | 1.20% |
| M300 | HV9 | 165 | 43.67 | 134.50 | 252.58 | 87.78 | 142.14 | 0.80% |
| MkVII | WS2 | 16 | 43.44 | 125.45 | 241.60 | 83.65 | 134.25 | 0.92% |
| MkVII | WS2 | 21 | 46.93 | 120.76 | 240.43 | 84.86 | 132.07 | 0.95% |

TABLE 7
Composition E (0.5% w/w PVP)

| Spray | | Spray | | | | | | |
|---|---|---|---|---|---|---|---|---|
| Pump | Insert | Volume (cc) | 10% | 50% | 90% | SMD | VMD | <15.5 μm |
| M300 | HV6 | 165 | 61.36 | 182.70 | 298.44 | 125.54 | 181.59 | 0.10% |
| M300 | HV9 | 165 | 71.67 | 212.05 | 328.87 | 140.63 | 206.45 | 0.23% |
| MkVII | WS2 | 16 | 69.49 | 220.03 | 342.10 | 139.25 | 214.35 | 0.37% |
| MkVII | WS2 | 21 | 67.86 | 202.95 | 305.29 | 132.73 | 196.94 | 0.35% |

TABLE 8
Composition F (0.7% w/w PVP)

| Spray | | Spray | | | | | | |
|---|---|---|---|---|---|---|---|---|
| Pump | Insert | Volume (cc) | 10% | 50% | 90% | SMD | VMD | <15.5 μm |
| M300 | HV6 | 165 | 58.02 | 218.17 | 380.12 | 132.04 | 218.10 | 0.09% |
| M300 | HV9 | 165 | 79.88 | 294.38 | 474.71 | 181.05 | 287.76 | 0.00% |
| MkVII | WS2 | 16 | 64.95 | 210.13 | 348.57 | 135.56 | 206.22 | 0.09% |
| MkVII | WS2 | 21 | 79.12 | 250.39 | 386.84 | 165.09 | 241.81 | 0.02% |

In Tables 4-8, the 10% column indicates that 10% of the liquid volume was the size shown (microns) or below. The 50% column shows half the liquid was above and half the liquid was below this particle size. The 90% column indicates that 90% of the liquid volume was below the particle size shown. The Sauter Mean Diameter (SMD) is an “average” diameter identifying the diameter of the droplet whose ratio of volume to surface area is the same as that of the entire spray. The Volume Mean Diameter (VMD) or Mass Median Diameter (MMD) shows the diameter of the drop where half of the volume of the spray contains droplets larger than the VMD and the other half contains smaller droplets. The last column in Tables 4-8 indicates the proportion of the liquid with droplet sizes of less than 15.5 microns.

As can be seen from the results shown in Tables 4-8 and FIGS. 1-10, while the different spray pumps and inserts had a minor effect on the particle sizes and rheology, the spray particle size and spray pattern in all cases were primarily impacted by the changing level of a suitable mucoadhesive, as PVP. These results show that even small changes in the amount of a mucoadhesive, such as PVP, in the spray composition can have dramatic effects on the particle size and the spray pattern.

### Example 4

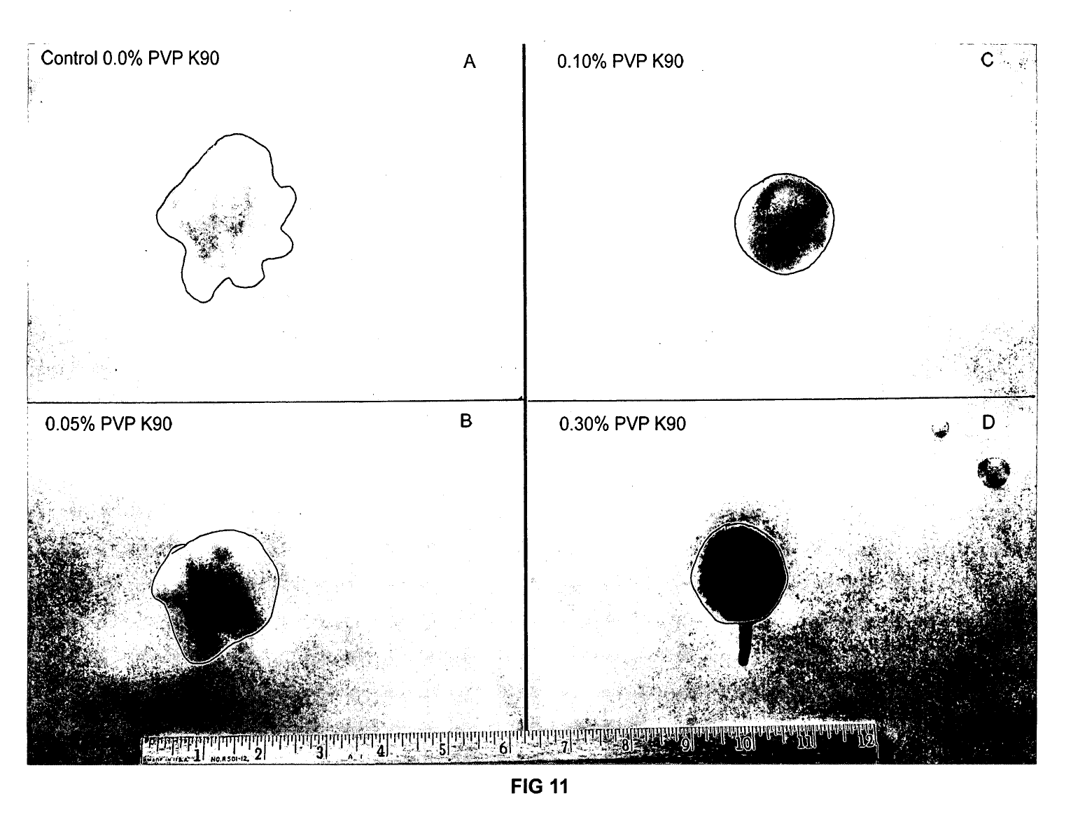

Compositions A-D shown in Table 3 were sprayed at a sheet from a distance of two inches using an Emsar 37MS fine mist spray with a 22/414 Nozzler in order to visually observe the spray pattern. The results of this test are shown in FIGS. 11A-11D.

The spray pattern of a composition without any mucoadhesive was irregular. As PVP was included in the composition, the spray stream become more organized, resulting in a regular spray pattern. As the PVP concentration reached 0.3% by weight, the stream became more concentrated, resulting in a denser deposition of the liquid. As shown in FIG. 11D, a part of the spray composition dripped after application.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An anesthetic spray composition comprising a topical anesthetic, a mucoadhesive polymer and a carrier.

2. The spray composition according to claim 1, wherein the topical anesthetic is benzocaine.

3. The spray composition according to claim 1, wherein the mucoadhesive polymer is polyvinyl pyrrolidone.

4. The spray composition according to claim 1, further comprising a solvent or solubilizer.

5. The spray composition according to claim 1, further comprising water.

6. The spray composition according to claim 1, wherein the mucoadhesive polymer comprises from about 0.08% to about 0.3% by weight of the spray composition.

7. The spray composition according to claim 1, further comprising at least one flavor and/or sweetener.

8. An anesthetic spray composition comprising benzocaine, polyvinyl pyrrolidone, ethanol, glycerin, polyethylene glycol castor oil and water.
